(19) United States
(12) Patent Application Publication
(10) Pub. No.: US 2007/0224643 A1
(43) Pub. Date: Sep. 27, 2007

(54) METHODS AND COMPOSITIONS FOR THE DIAGNOSIS OF DISEASES OF THE AORTA

(76) Inventors: Paul H. McPherson, Encinitas, CA (US); Ravi A. Vijayendran, Carlsbad, CA (US)

Correspondence Address: FOLEY & LARDNER LLP, P.O. BOX 80278, SAN DIEGO, CA, 92138-0278, US

(21) Appl. No.: 11/684,498

(22) Filed: Mar. 9, 2007

Related U.S. Application Data
(60) Provisional application No. 60/780,738, filed on Mar. 9, 2006.
(60) Provisional application No. 60/838,717, filed on Aug. 18, 2006.

(51) Int. Cl. G01N 33/53; G06F 19/00

(52) U.S. Cl. 435/7.1; 128/898; 702/19

(57) ABSTRACT

The present invention relates to methods and compositions for symptom-based differential diagnosis, prognosis, and determination of treatment regimens in subjects. In particular, the invention relates to the use of biomarkers, either individually or in combinations with one another to rule in or out diseases of the aorta and its branches, most particularly aortic aneurysm and/or aortic dissection, and for risk stratification in such conditions. Preferred markers include one or more of creatine kinase-BB (CK-BB), creatine kinase-MB (CK-MB), acidic calponin, basic calponin, B-type natriuretic peptide (BNP), NT-proBNP, proBNP, BNP₇₉₋₁₀₈, BNP₃₋₁₀₈, caldesmon, caspase-3, D-dimer, soluble elastin fragments, endothelial cell-selective adhesion molecule (ESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T, and preferred assays are configured to detect these markers.

### CROSS-REFERENCE TO RELATED PATENT APPLICATIONS

This application claims benefit of U.S. Provisional Application No. 60/780,738, filed Mar. 9, 2006, and U.S. Provisional Application No. 60/838,717 filed Aug. 18, 2006 each of which is incorporated by reference in its entirety, including all tables, figures, and claims.

### FIELD OF THE INVENTION

The present invention relates to the identification and use of diagnostic markers related to diseases of the aorta and its branches. In various aspects, the invention relates to methods and compositions for use in the diagnosis of aneurysms, and particularly dissecting aneurysms, and in the stratification of risk in such patients.

### BACKGROUND OF THE INVENTION

The following discussion of the background of the invention is merely provided to aid the reader in understanding the invention and is not admitted to describe or constitute prior art to the present invention.

The term “aneurysm” refers to a localized dilation of a blood vessel, particularly of the aorta or a peripheral artery. Aneurysms are related to arteriosclerosis, cystic medial necrosis, pathogen infection, aortitis, and trauma, each of which may contribute to weakening of the vessel wall. A familial component, particularly for certain aortic aneurysms, has also been reported. Common aneurysm sites include the abdominal aorta, the thoracic aorta, peripheral arteries such as the popliteal, iliac, and femoral arteries.

Abdominal aortic aneurysms may cause pain felt most prominently in the lumbosacral region. Even large aneurysms may be very difficult to detect on physical examination, especially in obese persons. Cross-sectional ultrasonography is the most cost-effective noninvasive method of evaluation. CT of the abdomen, particularly if performed with a contrast medium, or MRI can also determine aneurysmal size and anatomy but is more costly. Ordinary abdominal x-ray, particularly in a lateral position, may visualize the aneurysm due to calcification of the aneurysmal wall. Abdominal aortography is indicated if extension of the aneurysm above the renal arteries (present in about 10% of cases) is suspected.

Thoracic aortic aneurysms may cause pressure against, or erosion of, adjacent structures as the aorta enlarges. Clinical presentation may include pain; cough, wheezing, or hemoptysis from tracheal or bronchial compression or erosion; dysphagia from esophageal compression; or hoarseness from compression of the left recurrent laryngeal nerve. But as with abdominal aortic aneurysms, thoracic aneurysms may become quite large while remaining asymptomatic. Thoracic aortic aneurysms can usually be seen on chest x-ray, CT and MRI. Transthoracic ultrasonography is accurate in sizing aneurysms of the ascending but not the descending aorta, whereas transesophageal ultrasonography appears accurate in sizing aneurysms of both. Contrast or magnetic resonance aortography is indicated for most thoracic aneurysms being evaluated for surgical resection.

Dissection (rupture) of an aortic aneurysm carries a high risk of death. Aortic dissection is characterized by the rapid development of an intimal flap separating the true and false channel of the aorta. In addition to a tear in the intimal layer there is a formation and propagation of a subintimal hematoma. The advancing column of blood forms a false channel within the aorta, which usually extends distally and, less commonly, proximally from the initial intimal tear. Dissection occurs within the layers of the vascular media, and may rupture through the adventitia or back through the intima. The false channel may reenter the true aortic lumen at any point. The blood supply of any tributary artery of the aorta may be compromised, resulting in ischemia of the served portion of the body. Death often follows due to rupture of the aorta, usually into the pericardial cavity or left pleural space. When untreated, about 33% of patients die within the first 24 hours and 50% die within 48 hours. Approximately 75% of patients with undiagnosed ascending aortic dissection die within two weeks.

Dissection may originate anywhere along the aorta, but the most common sites are the proximal ascending aorta (within 5 cm of the aortic valve) and the descending thoracic aorta (just beyond the origin of the left subclavian artery). Rarely, dissection is confined to individual peripheral (e.g., coronary or carotid) arteries. Degenerative changes in the smooth muscle and elastic tissue of the aortic wall underlie most cases. The most common conditions associated with dissection are hypertension (present in >⅔ of cases and is especially prevalent with distal dissection), hereditary connective tissue disorders (e.g., Marfan's and Ehlers-Danlos syndromes), congenital cardiovascular abnormalities (e.g., coarctation of the aorta, patent ductus arteriosus, bicuspid aortic valve), arteriosclerosis, trauma, and granulomatous arteritis.

The primary symptom reported in aortic dissection is pain, which is almost always present. The pain is typically abrupt in onset, excruciating, and is often described as tearing or ripping. The most common location is the precordium, but pain in the interscapular area is frequent, especially with dissection involving the descending thoracic aorta. The pain of aortic dissection frequently migrates from the original site as the dissection extends along the aorta. Occasionally, dissection presents with symptoms related to an acute stroke, myocardial infarction or intestinal infarction; paraparesis or paraplegia from interruption of the blood supply to the spinal cord; or an ischemic limb). Such clinical symptoms often mimic arterial embolism.

### BRIEF SUMMARY OF THE INVENTION

The present invention relates to the identification and use of markers for the detection and risk stratification of diseases of the aorta and its branches. In various aspects, the invention relates to methods and compositions for use in the diagnosis of aneurysms, and particularly dissecting aneurysms, and in the stratification of risk in such patients. The methods and compositions described herein can meet the need in the art for a rapid, sensitive and specific diagnostic assay to be used in the diagnosis and differentiation in such conditions. Moreover, the methods and compositions of the present invention can also be used to facilitate the treatment of patients and the development of additional diagnostic and/or prognostic indicators.

In various aspects, the invention relates to materials and procedures for identifying markers that are associated with the diagnosis, prognosis, or differentiation of diseases of the aorta and its branches in a patient; to using such markers in diagnosing and treating a patient and/or to monitor the course of a treatment regimen; to using such markers to identify subjects at risk for one or more adverse outcomes related to diseases of the aorta and its branches; and for screening compounds and pharmaceutical compositions that might provide a benefit in treating or preventing such conditions, e.g., for efficacy.

In a first aspect, the invention discloses methods for determining a diagnosis in a subject suspected of having an aneurysm of the aorta and/or one or more of its branches (referred to herein as an “aortic aneurysm”); and/or determining a prognosis in a subject diagnosed with an aortic aneurysm. In a related aspect, the invention discloses methods for determining a diagnosis in a subject suspected of having a dissecting aneurysm in the aorta and/or one or more of its branches (referred to herein as an “aortic dissection”); and/or determining a prognosis in a subject diagnosed with an aortic dissection.

These methods comprise analyzing a test sample obtained from a subject for using one or more assays that detect the presence or amount of one or more, and most preferably a plurality of, markers independently selected from the group consisting of markers related to blood pressure regulation, markers related to inflammation, markers related to apoptosis, markers related to reactive oxygen species, markers related to myocardial injury, markers related to pulmonary injury, markers related to vascular injury, and markers related to coagulation and hemostasis. Exemplary markers for use in these methods are described hereinafter. While such markers may be used to rule in or out aortic aneurysm and/or aortic dissection or to assign a prognosis in these conditions, such markers may also be used to rule in or out other conditions that are within the differential diagnosis of aortic aneurysm and/or aortic dissection.

The assay results obtained are used to diagnose aortic aneurysm and/or aortic dissection in a subject, are used to provide a prognosis for a subject having aortic aneurysm and/or aortic dissection, or to both diagnose and provide a prognosis for a subject.

In preferred embodiments, these methods comprise analyzing a test sample obtained from a subject for using one or more assays that detect the presence or amount of one or more markers selected from the group consisting of creatine kinase-BB (CK-BB), creatine kinase-MB (CK-MB), acidic calponin, basic calponin, neutral calponin, soluble junction adhesion molecule-1 (sJAMl), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, cardiac troponin I (free and/or complexed), and cardiac troponin T (free and/or complexed). In certain embodiments, a panel comprises a plurality of markers, comprising 2, 3, 4, 5, 6, 7, 8, 9, 10, 15, 20, or more, markers, and such panels may comprise 1, 2, 3, 4, 5, 6, 7, 8, 9, 10, 15, 20, or more, of the preferred markers recited in this paragraph.

In various preferred embodiments, these methods comprise analyzing a test sample obtained from a subject using assays that detect the presence or amount of one or more markers selected from the group consisting of BNP, NT-proBNP, proBNP, creatine kinase-BB (CK-BB), creatine kinase-MB (CK-MB), acidic calponin, basic calponin, neutral calponin, soluble junction adhesion molecule-2 (sJAM2), soluble junction adhesion molecule-3 (sJAM3), caldesmon, caspase-3, sESAM, heart-type fatty acid binding protein, and TIMP-1. Such panels may comprise analyzing a test sample obtained from a subject using assays that detect the presence or amount of 2, 3, 4, 5, or 6 of the preferred markers recited in this paragraph, and may optionally comprise analyzing a test sample obtained from a subject using assays that detect the presence or amount of one or more additional markers as described herein. Particularly preferred embodiments comprise analyzing a test sample obtained from a subject using assays that detect the presence or amount of one or more markers selected from the group consisting of creatine kinase-BB (CK-BB), acidic calponin, basic calponin, neutral calponin, sJAM2, sJAM3, caldesmon, caspase-3, and sESAM, and may optionally comprise analyzing a test sample obtained from a subject using assays that detect the presence or amount of one or more additional markers as described herein.

In certain embodiments, concentrations of individual markers can be compared to one or more levels (that is, a “tnreshold”) that is associated with the particular diagnosis and/or prognosis of interest. By correlating each of the subject's selected marker level(s) to threshold(s) for each marker of interest, the subject may be assigned to a diagnostic group (e.g., suffering from one of these conditions, or not suffering from one of these conditions). Similarly, by correlating the subject's marker levels to prognostic thresholds for each marker, the probability or risk that the subject will suffer one or more future adverse outcomes may be determined. Thresholds are typically established using Receiver Operating Characteristic (“ROC”) analysis of individual markers to select an appropriate level of specificity and sensitivity.

In other embodiments, particular thresholds are not relied upon to determine if one or more marker levels obtained from a subject are indicative of a particular diagnosis or prognosis. Rather, the present invention may utilize an evaluation of the entire profile of a panel of markers. For example, by plotting Receiver Operating Characteristic (“ROC”) curves for the sensitivity of a particular panel of markers versus 1-(specificity) for the panel at various marker levels, a profile of marker measurements from a subject may be considered together to provide a global probability (a “panel response” expressed either as a numeric score or as a percentage risk) that the symptom(s) observed in an individual are caused by a particular underlying disease. In such embodiments, an increase (or decrease) in a certain subset of markers may be sufficient to indicate a particular diagnosis in one patient, while an increase (or decrease) in a different subset of markers may be sufficient to indicate the same or a different diagnosis in another patient. Methods for performing such analyses are described hereinafter.

In yet other embodiments, multiple determinations of markers can be made, and a temporal change in the markers can be used to rule in or out one or more particular etiologies for observed symptom(s). For example, one or more markers may be determined at an initial time, and again at a second time, and the change (or lack thereof) in the marker level(s) over time determined. In such embodiments, an increase in the marker from the initial time to the second time may be diagnostic of a particular disease (e.g., aortic aneurysm and/or aortic dissection) underlying one or more symptoms, a particular prognosis, etc. Likewise, a decrease in the marker from the initial time to the second time may be indicative of a particular disease underlying one or more symptoms, a particular prognosis, etc. Temporal changes in one or more markers may also be used together with single time point marker levels to increase the discriminating power of marker panels. In yet another alternative, a “panel response” may be treated as a marker, and temporal changes in the panel response may be indicative of a particular disease underlying one or more symptoms, a particular prognosis, etc.

Receiver Operating Characteristic curves, or “ROC” curves, are typically calculated by plotting the value of a variable versus its relative frequency in “normal” and “disease” populations, where “normal” and “disease” simply indicates the absence and presence of some characteristic of interest. For any particular marker, a distribution of marker levels for subjects with and without a “disease” will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

One or more of the markers described herein may be combined in a single assay or device that performs assays configured to detect a plurality of markers. In such embodiments, certain markers measured by a device or instrument may be used to diagnose aortic aneurysm, while the same or different markers measured by the same device may be used to diagnose aortic dissection. Each condition may be diagnosed with sets of markers that may comprise unique markers, or may include markers that overlap with one or both of the other sets. Markers may also be commonly used for multiple purposes by, for example, applying a different set of analysis parameters (e.g., a threshold or a different weighting factor) to the marker(s) for the different purpose(s). For example, a marker at one concentration or weighting may be used, alone or as part of a larger panel, to indicate a diagnosis of aortic aneurysm and/or aortic dissection, and the same marker at a different concentration or weighting may be used, alone or as part of the same or a different panel, to indicate prognosis associated with the diagnosis.

The sensitivity and specificity of a diagnostic and/or prognostic test depends on more than just the analytical “quality” of the test—they also depend on the definition of what constitutes an abnormal result. In practice, In preferred embodiments, markers and/or marker panels are selected to exhibit at least 75% sensitivity, more preferably at least 80% sensitivity, even more preferably at least 85% sensitivity, still more preferably at least 90% sensitivity, and most preferably at least 95% sensitivity, combined with at least 75% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to ±5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to ±5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to ±5% of a given measurement.

One or more markers may lack predictive value when considered alone, but when used as part of a panel, such markers may be of great value in determining a particular diagnosis/prognosis. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker alone is sufficient to signal a positive result, but only signals a result in combination with one or more other markers in the panel.

While exemplary panels are described herein, one or more markers may be replaced, added, or subtracted from these exemplary panels while still providing clinically useful results. Panels may comprise both specific markers of aortic aneurysm and/or aortic dissection; and/or non-specific markers (e.g., markers that are increased or decreased due to inflammation, regardless of the cause; markers that are increased or decreased due to changes in hemostasis, regardless of the cause, etc.). While non-specific (and/or specific) markers may not individually be diagnostic of aortic aneurysm and/or aortic dissection, a particular “fingerprint” pattern of changes may, in effect, act as a specific indicator of a disease or condition. As discussed above, that pattern of changes may be obtained from a single sample, or may optionally consider temporal changes in one or more members of the panel (or temporal changes in a panel response value).

In a particularly preferred embodiment, the marker(s) used in the diagnostic and prognostic methods and compositions described herein comprises at least one, and preferably two or more, markers related to coagulation and hemostasis. Thus, preferred assays and assay devices are configured to detect one or more such markers. Particularly preferred markers related to coagulation and hemostasis include those selected from the group consisting of plasmin, thrombin, antithrombin-III, fibrinogen, one or more forms of von Willebrand factor, D-dimer, PAI-1, soluble urokinase plasminogen activator surface receptor (uPAR), Protein C, soluble endothelial protein C receptor (EPCR), TAFI, fibrinopeptide A, plasmin alpha 2 antiplasmin complex, platelet factor 4, platelet-derived growth factor, P-selectin, prothrombin fragment 1+2, B-thromboglobulin, thrombin antithrombin III complex, thrombomodulin, thrombus precursor protein, tissue factor, tissue factor pathway inhibitor-α, and tissue factor pathway inhibitor-β, or markers related thereto. Most preferred markers are selected from the group consisting of D-dimer, uPAR, TpP, and one or more forms of von Willebrand factor, or markers related thereto. Most preferred are assays that detect the presence or amount of D-dimer.

In another particularly preferred embodiment, the marker(s) used in the diagnostic and prognostic methods and compositions described herein comprises at least one, and preferably two or more, markers related to blood pressure regulation. Thus, preferred assays and assay devices are configured to detect one or more such markers. Particularly preferred markers related to blood pressure regulation are selected from the group consisting of atrial natriuretic peptide (“ANP), pro-ANP, B-type natriuretic peptide (“BNP”), NT-pro BNP, pro-BNP C-type natriuretic peptide, urotensin II, urocortin I, urocortin II, urocortin III, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or markers related thereto. Most preferred are assays that detect the presence or amount of one or more markers selected from the group consisting of BNP, proBNP, NT-proBNP, BNP₇₉₋₁₀₈, and BNP₃₋₁₀₈, or markers related thereto.

In still another particularly preferred embodiment, marker(s) used in the diagnostic and prognostic methods and compositions described herein comprises at least one, and preferably two or more, markers related to inflammation. Thus, preferred assays and assay devices are configured to detect one or more such markers. Particularly preferred markers are selected from the group consisting of acute phase reactants, cell adhesion molecules such as vascular cell adhesion molecule (“VCAM”), intercellular adhesion molecule-1 (“ICAM-1”), intercellular adhesion molecule-2 (“ICAM-2”), and intercellular adhesion molecule-3 (“ICAM-3”), C-reactive protein, HMG-1 (also known as HMGB1), interleukins such as IL-1β, IL-6, IL-8, interleukin-1 receptor agonist, monocyte chemotactic protein-1, caspase-3, lipocalin-type prostaglandin D synthase, mast cell tryptase, eosinophil cationic protein, KL-6, haptoglobin, tumor necrosis factor a, tumor necrosis factor β, Fas ligand, soluble Fas (Apo-1), TRAIL, TWEAK, fibronectin, macrophage migration inhibitory factor (MIF), and vascular endothelial growth factor (“VEGF”), or markers related thereto.

Acute phase reactants may be selected from the group consisting of hepcidin, HSP-60, HSP-65, HSP-70, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, inter-α-inhibitors, e-selectin, glutathione-S-transferase, hypoxia-inducible factor-1α, inducible nitric oxide synthase (“I-NOS”), intracellular adhesion molecule, lactate dehydrogenase, matrix metalloproteinase-9 (“MMP-9”), monocyte chemoattractant peptide-1 (“MCP-1”), n-acetyl aspartate, prostaglandin E2, receptor activator of nuclear factor (“RANK”) ligand, TNF receptor superfamily member 1A, and cystatin C, or markers related thereto.

In still another particularly preferred embodiment, marker(s) used in the diagnostic and prognostic methods and compositions described herein comprises at least one, and preferably two or more, markers related to reactive oxygen species. Thus, preferred assays and assay devices are configured to detect one or more such markers. The marker(s) may be selected from the group consisting of superoxide dismutase, glutathione, α-tocopherol, ascorbate, inducible nitric oxide synthase, lipid peroxidation products, nitric oxide, mycloperoxidase, and breath hydrocarbons (preferably ethane), or markers related thereto. Most preferred are assays that detect the presence or amount of myelopcroxidase.

Additional markers and/or marker classes may be used in such panels. For example, the inflammatory response and resulting effects on capillaries and reduced oxygenation of tissues implicate one or more markers related to the acute phase response, one or more markers related to vascular tissues, and one or more tissue-specific (e.g., neural-specific) markers, the levels of which are increased in ischemic conditions. Preferably, one or more markers selected from the group consisting of α-2 actin, basic calponin 1, acidic calponin, β-1 integrin, caldesmon, cysteine rich protein-2 (“CRP 2” or “CSRP 2”), elastin, soluble elastin fragments, fibrillin 1, latent transforming growth factor beta binding protein 4 (“LTBP 4”), smooth muscle myosin, smooth muscle myosin heavy chain, and transgelin, or markers related thereto (referred to collectively as “markers related to vascular tissue”) may be included in such a panel. Thus, preferred assays and assay devices are configured to detect one or more such markers.

Additional marker classes, such as markers related to myocardial injury, markers related to neural tissue injury, markers related to pulmonary injury, etc., are described hereinafter and may also be used in such panels. Preferred markers related to pulmonary injury may be selected from the group consisting of neutrophil elastase, KL-6, LAMP 3, LAMP3, pulmonary surfactant protein A, pulmonary surfactant protein B, pulmonary surfactant protein C, pulmonary surfactant protein D, phospholipase D, PLA2G5, SFTPC, HT156, and HT11280, or markers related thereto. Thus, preferred assays and assay devices are configured to detect one or more such markers.

Preferred markers related to myocardial injury may be selected from the group consisting of cardiac troponin I (free and/or complexed), cardiac troponin T (free and/or complexed), annexin V, B-enolase, CK-MB, glycogen phosphorylase-BB, heart type fatty acid binding protein, phosphoglyceric acid mutase, and S-100ao, or markers related thereto. Most preferred are assays that detect the presence or amount of cardiac troponin I (free and/or complexed) and/or cardiac troponin T (free and/or complexed). Thus, preferred assays and assay devices are configured to detect one or more such markers.

The markers described herein may be variously combined to provide suitable marker panels. In various embodiments, the plurality of markers comprises at least one marker related to coagulation and hemostasis and at least one marker related to inflammation; the plurality of markers comprises at least one marker related to blood pressure regulation and at least one marker related to coagulation and hemostasis; the plurality of markers comprises at least one marker related to blood pressure regulation, at least one marker related to inflammation, and at least one marker related to coagulation and hemostasis; the plurality of markers comprises at least one marker related to apoptosis and at least one marker related to coagulation and hemostasis; the plurality of markers comprises at least one marker related to reactive oxygen species and at least one marker related to coagulation and hemostasis; the plurality of markers comprises at least one marker related to myocardial injury and at least one marker related to coagulation and hemostasis; the plurality of markers comprises at least one marker related to pulmonary injury and at least one marker related to coagulation and hemostasis; the plurality of markers comprises at least one marker related to blood pressure regulation, at least one marker related to myocardial injury, and at least one marker related to coagulation and hemostasis; the plurality of markers comprises at least one marker related to blood pressure regulation, at least one marker related to pulmonary injury, and at least one marker related to coagulation and hemostasis; the plurality of markers comprises at least one marker related to blood pressure regulation, at least one marker related to apoptosis, and at least one marker related to coagulation and hemostasis; the plurality of markers comprises at least one marker related to blood pressure regulation, at least one marker related to apoptosis, at least one marker related to inflammation, and at least one marker related to coagulation and hemostasis; or the plurality of markers comprises at least one marker related to blood pressure regulation, at least one marker related to myocardial injury, and at least one marker related to coagulation and hemostasis. These combinations are not meant to be limiting.

In related aspects, the present invention relates to methods for identifying marker panels for use in the foregoing methods. In developing a panel of markers useful in diagnosis and/or prognosis, data for a number of potential markers may be obtained from a group of subjects by testing for the presence or level of certain markers. The group of subjects may then be divided into sets. For example, a first set includes subjects who have been confirmed as having a disease or, more generally, being in a first condition state. The confirmation of this condition state may be made through a more rigorous and/or expensive testing. A second set of subjects is selected from those who do not fall within the first set. [0038] The data obtained from subjects in these sets includes levels of a plurality of markers described herein. Preferably, data for the same set of markers is available for each patient. Exemplary markers are described herein. Actual known relevance of the marker(s) to the disease of interest is not required. Methods for comparing these subject sets for relevance of one or more markers is described hereinafter. Embodiments of the methods and systems described herein may be used to determine which of the candidate markers are most relevant to the diagnosis of the disease or condition or of a given prognosis.

In another aspect, the invention relates to methods for determining a treatment regimen for use in a subject exhibiting aortic aneurysm and/or aortic dissection. The methods preferably comprise performing the methods described herein to rule in or out aortic aneurysm and/or aortic dissection; and/or to assign a prognosis to a subject diagnosed with aortic aneurysm and/or aortic dissection. One or more treatment regimens can then be selected, based on the condition and/or prognosis assigned to the subject.

In a further aspect, the invention relates to kits to rule in or out aortic aneurysm and/or aortic dissection; and/or to assign a prognosis to a subject diagnosed with aortic aneurysm and/or aortic dissection. These kits preferably comprise devices and reagents for performing an assay that detects one or more of the markers described herein, and instructions for relating the assay(s) to rule in or out aortic aneurysm and/or aortic dissection; and/or to assign a prognosis to a subject diagnosed with aortic aneurysm and/or aortic dissection. Such kits preferably contain sufficient reagents to perform at least one, and preferably two or more such determinations, and/or Food and Drug Administration (FDA)-approved labeling. As discussed above, such kits preferably comprise reagents configured to detect the presence or amount of one or more markers selected from the group consisting of creatine kinase-BB (CK-BB), creatine kinase-MB (CK-MB), acidic calponin, basic calponin, B-type natriuretic peptide (BNP), NT-proBNP, proBNP, BNP₇₉₋₁₀₈, BNP₃₋₁₀₈, caldesmon, caspase-3, D-dimer, soluble elastin fragments, endothelial cell-selective adhesion molecule (ESAM) extracellular domain, fibrillin-1, heart-type fatty acid binding protein, MMP-9, myeloperoxidase, myoglobin, smooth muscle myosin, smooth muscle myosin heavy chain, TIMP-1, cardiac troponin I (free and/or complexed), and cardiac troponin T (free and/or complexed). Such reagents include antibodies for binding the selected marker(s), and most preferably include reagents for detecting the binding of selected marker(s) to such antibodies in sandwich and/or competitive immunoassay formats.

In yet a further aspect, the invention relates to devices to rule in or out aortic aneurysm and/or aortic dissection; and/or to assign a prognosis to a subject diagnosed with aortic aneurysm and/or aortic dissection. Such devices preferably contain a plurality of diagnostic zones, each of which is configured to provide a signal related to the presence or amount of a particular marker of interest. Such devices may be referred to as “arrays” or “microarrays.” Following reaction of a sample with the devices, a signal is generated from the diagnostic zone(s), which may then be correlated to the presence or amount of the markers of interest. Numerous suitable devices are known to the skilled artisan. Such devices preferably comprise reagents configured to detect the presence or amount of one or more markers selected from the group consisting of creatine kinase-BB (CK-BB), creatine kinase-MB (CK-MB), acidic calponin, basic calponin, B-type natriuretic peptide (BNP), NT-proBNP, proBNP, BNP₇₉₋₁₀₈, BNP₃₋₁₀₈, caldesmon, caspase-3, D-dimer, soluble elastin fragments, endothelial cell-selective adhesion molecule (ESAM) extracellular domain, fibrillin-1, heart-type fatty acid binding protein, MMP-9, myeloperoxidase, myoglobin, smooth muscle myosin, smooth muscle myosin heavy chain, TIMP-1, cardiac troponin I (free and/or complexed), and cardiac troponin T (free and/or complexed). Such reagents include antibodies for binding the selected marker(s), and most preferably include reagents for detecting the binding of selected marker(s) to such antibodies in sandwich and/or competitive immunoassay formats.

### DETAILED DESCRIPTION OF THE INVENTION

The present invention relates in part to methods and compositions for diagnosis, prognosis, and determination of treatment regimens in subjects. In particular, the invention relates to methods and compositions selected to rule in or out one or more diseases of the aorta and its branches, most preferably aortic aneurysm and/or aortic dissection; and or to assign a prognosis to a subject diagnosed with one or more diseases of the aorta and its branches, most preferably aortic aneurysm and/or aortic dissection.

Differential diagnosis refers to methods for diagnosing the particular disease(s) underlying the symptoms in a particular subject, based on a comparison of the characteristic features observable from the subject to the characteristic features of those potential diseases. Depending on the breadth of diseases that must be considered in the differential diagnosis, the types and number of tests that must be ordered by a clinician can be quite large. The clinician must then integrate information obtained from a battery of tests, leading to a clinical diagnosis that best represents the range of symptoms and/or diagnostic test results obtained for the subject.

Patients presenting for medical treatment often exhibit one or a few primary observable changes in bodily characteristics or functions that are indicative of disease. Often, as in the case of aortic aneurysm and aortic dissection, these “symptoms” are nonspecific, in that a number of potential diseases can present the same observable symptom or symptoms.

The present invention describes methods and compositions that can assist in the differential diagnosis of one or more nonspecific symptoms by providing diagnostic markers that are designed to rule in or out one, and preferably a plurality, of possible etiologies for the observed symptoms. Symptom-based differential diagnosis described herein can be achieved using panels of diagnostic markers designed to distinguish between possible diseases that underlie a nonspecific symptom observed in a patient.

### DEFINITIONS

The term “marker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments. Markers can also include other measurable characteristics including results from blood pressure measurements, temperature measurements, pulse oximetry measurements, patient history, radiography, electrocardiogram, exercise treadmill testing, blood chemistry analysis, echocardiography, bronchoprovocation testing, spirometry, pulse oximetry, esophageal pH monitoring, laryngoscopy, computed tomography, histology, cytology, magnetic resonance imaging, etc. Similarly, markers can also include clinical “scores” such as a pre-test probability assignment, a Glasgow Aneurysm Score, etc.

Preferably, the methods described hereinafter utilize one or more markers that are derived from the subject. The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. Most preferred are protein and polypeptide markers. The presence, absence, amount, or change in amount of one or more markers may indicate that a particular disease is present, or may indicate that a particular disease is absent. Additionally, “non-subject-derived markers” may also be used. Such markers are not derived from the subject as defined herein, but rather that are characteristics of the subject observable by the artisan. Such markers are discussed above, and can include various measureable characteristics and/or clinical scores. This list is not meant to be limiting.

The term “related marker” as used herein refers to one or more fragments of a particular subject-derived marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-1 08 of this precursor, which may be referred to as BNP₇₇₋₁₀₈. The remaining residues 1-76 are referred to hereinafter as BNP₁₋₇₆. Additionally, related markers may be the result of covalent modification of the parent marker, for example by oxidation of methionine residues, ubiquitination, cysteinylation, phosphorylation, nitrosylation, glycosylation, etc.

The sequence of the 1 08 amino acid BNP precursor pro-BNP (BNP₁₋₁₀₈) is as follows, with mature BNP (BNP₇₇₋₁₀₈) underlined:

| (SEQ ID NO: 1) | |
|---|---|
| HPLGSPGSAS DLETSGLQEQ RNHLQGKLSE LQVEQTSLEP LQESPRPTGV | 50 |
| WKSREVATEG IRGHRKMVLY TLRAPRSPKM VQGSGCFGRK MDRISSSSGL | 100 |
| GCKVLRRH. | 108 |

BNP1-108 is synthesized as a larger precursor pre-pro-BNP having the following sequence (with the “pre” sequence shown in bold):

| (SEQ ID NO: 2) | |
|---|---|
| MDPQTAPSRA LLLLLFLHLA FLGGRSHPLG SPGSASDLET SGLQEQRNHL | 50 |
| QGKLSELQVE QTSLEPLQES PRPTGVWKSR EVATEGIRGH RKMVLYTLPA | 100 |
| PRSPKMVQGS GCFGRKMDRI SSSSGLGCKV LRRH. | 134 |

While mature BNP itself may be used as a marker in the present invention, the prepro-BNP, BNP1-108 and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆ and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. Ser. No. 10/419,059, filed Apr. 17, 2003, which is hereby incorporated by reference in its entirety including all tables, figures and claims. Preferred BNP-related molecules are proBNP, NT-proBNP, BNP₇₉₋₁₀₈, and BNP₃₋₁₀₈.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described for BNP.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for BNP, and a significant amount (eg., 50%) of the biologically active BNP that had been present has now been degraded into an inactive form. An immunoassay formulated with antibodies that bind a region common to the biologically active BNP and the inactive fragment(s) will overestimate the amount of biologically active BNP present in the sample by 2-fold, potentially resulting in a “false positive” result. Overestimation of the biologically active form(s) present in a sample may also have serious consequences for patient management. Considering the BNP example again, the BNP concentration may be used to determine if therapy is effective (e.g., by monitoring BNP to see if an elevated level is returning to normal upon treatment). The same “false positive” BNP result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of troponin may not detect all of the cardiac-specific isoform of interest.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care or are being evaluated in a medical setting. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high (or low) level in samples obtained from such patients, the level may signal that the patient is at an increased probability for experiencing a future outcome in comparison to a similar patient exhibiting a lower (or higher) marker level. A level or a change in level of a prognostic indicator, which in turn is associated with an increased probability of morbidity or death, is referred to as being “associated with an increased predisposition to an adverse outcome” in a patient.

The term “correlating,” as used herein in reference to the use of diagnostic and prognostic markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels is correlated to a global probability or a particular outcome using ROC curves.

The terms “diagnosis” and “prognosis” do not refer to the ability to determine the presence or absence of a particular disease with 100% accuracy, or even that a given course or outcome is more likely to occur than not. Instead, the skilled artisan will understand that the term “diagnosis” and “prognosis” refers to an increased probability that a certain disease is present in the subject. In preferred embodiments, a diagnosis indicates about a 5% increased chance that a disease is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to ±2%.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Exemplary Markers

CK-BB

Creatine kinase (CK) is a cytosolic enzyme that catalyzes the reversible formation of ADP and phosphocreatine from ATP and creatine. CK is a homo- or heterodimer composed of M and B chains. The so-called brain-specific CK isoform (CK-BB) is an 85 kDa cytosolic protein that accounts for approximately 95% of the total brain CK activity. It is also present in significant quantities in cardiac tissue, intestine, prostate, rectum, stomach, smooth muscle, thyroid uterus, urinary bladder, and veins (Johnsson, P. J., Cardiothorac. Vasc. Anesth. 10:120-126, 1996). The normal serum concentration of CK-BB is <10 ng/ml (120 pM).

CK-MB

CK-MB is the isoform that is most specific for cardiac tissue, but it is also present in skeletal muscle and other tissues. The normal plasma concentration of CK-MB is <5 ng/ml. The plasma CK-MB concentration is significantly elevated in patients with AMI. Plasma CK-MB is not elevated in patients with stable angina, and investigation into plasma CK-MB concentration elevations in patients with unstable angina have yielded mixed results (Thygesen, K. et al., Eur. J. Clin. Invest. 16:1-4, 1986; Koukkunen, H. et al., Ann. Med. 30:488-496, 1998; Bertinchant, J. P. et al., Clin. Biochem. 29:587-594, 1996; Benamer, H. et al., Am. J. Cardiol. 82:845-850, 1998; Norregaard-Hansen, K. et al., Eur. Heart J. 13:188-193, 1992). CK-MB is released into the bloodstream following cardiac cell death. The plasma concentration of CK-MB in patients with AMI is significantly elevated 4-6 hours after onset, peaks between 12-24 hours, and returns to baseline after 3 days. The release kinetics of CK-MB associated with unstable angina may be similar.

Acidic Calponin

Acidic calponin, also called calponin-3 (Human precursor: Swiss-Prot Q15417), is a thin filament-associated protein expressed in both smooth muscle and in non-muscle cells. It is suggested to be involved in regulation of smooth muscle contraction, as it binds to F-actin and inhibits actin-activated myosin ATPase activity in a calcium/calmodulin-dependent manner.

Basic Calponin

Basic calponin, also known as calponin-l and calponin Hi (Human precursor: Swiss-Prot P51911), is also a thin filament-associated protein that is implicated in the regulation and modulation of smooth muscle contraction. It is capable of binding to actin, calmodulin, troponin C and tropomyosin.

Neutral Calponin

Neutral calponin, also known as calponin-2 (Human precursor: Swiss-Prot Q99439) is still another thin filament-associated protein that is implicated in the regulation and modulation of smooth muscle contraction. It is capable of binding to actin, calmodulin, troponin C and tropomyosin.

Myoglobin

Myoglobin (Human precursor: Swiss-Prot P02144) is a single-chain globular protein of 153 amino acids, contains a heme prosthetic group, and is the primary oxygen-carrying pigment of muscle tissues. Myoglobin release into the blood is used as a sensitive marker for muscle injury, and is considered an early marker of myocardial infarction.

Cardiac troponins

Troponin I (TnI) is a 25 kDa inhibitory element of the troponin complex, found in all striated muscle tissue. TnI binds to actin in the absence of Ca²⁺, inhibiting the ATPase activity of actomyosin. A TnI isoform that is found in cardiac tissue (cTnI) is 40% divergent from skeletal muscle TnI, allowing both isoforms to be immunologically distinguished. The normal plasma concentration of cTnI is <0.1 ng/ml (4 pM). The plasma cTnI concentration is elevated in patients with acute myocardial infarction. Investigations into changes in the plasma cTnI concentration in patients with unstable angina have yielded mixed results, but cTnI is not elevated in the plasma of individuals with stable angina (Benamer, H. et al., Am. J Cardiol. 82:845-850, 1998; Bertinchant, J. P. et al., Clin. Biochem. 29:587-594, 1996; Tanasijevic, M. J. et al., Clin. Cardiol. 22:13-16, 1999; Musso, P. et al., J. Ital. Cardiol. 26:1013-1023, 1996; Holvoet, P. et al., JAMA 281:1718-1721, 1999; Holvoet, P. et al., Circulation 98:1487-1494, 1998). The mixed results associated with unstable angina suggest that cTnI may be useful in determining the severity of unstable angina because the extent of myocardial ischemia is directly proportional to unstable angina severity. The plasma cTnI concentration may be elevated in conjunction with cardiac trauma, congestive heart failure, and cardiac surgery, non-ischemic dilated cardiomyopathy, muscular disorders, CNS disorders, HIV infection, chronic renal failure, sepsis, lung disease, and endocrine disorders (Khan, I. A. et al., Am. J. Emerg. Med. 17:225-229, 1999). This apparent non-specificity may be related to the quality and specificity of the antibodies used in the immunoassay. cTnI is released into the bloodstream following cardiac cell death. The plasma concentration of cTnI in patients with AMI is significantly elevated 4-6 hours after onset, peaks between 12-16 hours, and can remain elevated for one week. The release kinetics of cTnI associated with unstable angina may be similar. The measurement of specific forms of cardiac troponin, including free cardiac troponin I and complexes of cardiac troponin I with troponin C and/or T may provide the user with the ability to identify various stages of ACS.

Free and complexed cardiac-troponin T may be used in a manner analogous to that described above for cardiac troponin I. Cardiac troponin T complex may be useful either alone or when expressed as a ratio with total cardiac troponin I to provide information related to the presence of progressing myocardial damage. Ongoing ischemia may result in the release of the cardiac troponin TIC complex, indicating that higher ratios of cardiac troponin TIC:total cardiac troponin I may be indicative of continual damage caused by unresolved ischemia.

JAM2

Junction Adhesion Molecule-2 (Human precursor: Swiss-Prot P57087) is a type I membrane protein expressed in the heart, among other tissues. It is predominantly expressed on high endothelial venules, but is also present on the endothelia of other vessels. It is localized to tight junctional complexes. Soluble forms may be generated through proteolysis or alternative splicing events, resulting in a non-membrane-bound form containing all or a portion of the extracellular domain of JAM2. The JAM2 protein is 35% identical to the JAM1 protein, and assays may be developed that detect both JAM1 and JAM2, or that are specific for JAM1 or JAM2 relative to the other molecule.

JAM3

Junction Adhesion Molecule-3 (Human precursor: Swiss-Prot Q9BX67) is a type I membrane protein expressed in the heart, among other tissues. It is most highly expressed in placenta, brain and kidney. It is localized to tight junctional complexes. JAM3 interacts strongly with JAM2. Soluble forms may be generated through proteolysis or alternative splicing events, resulting in a non-membrane-bound form containing all or a portion of the extracellular domain of JAM3. The JAM3 protein is more than 30% identical to JAM2 and JAM 1, and assays may be developed that detect JAM1, JAM2, and JAM3, or that are specific for JAM1, JAM2, or JAM3 relative to the other molecules.

Caldesmon

Caldesmon (Human precursor: Swiss-Prot Q05682) is an actin- and myosin-binding protein implicated in the regulation of actomyosin interactions in smooth muscle and nonmuscle cells. Caldesmon interacts with actin, myosin, two molecules of tropomyosin and with calmodulin.

Caspase-3

Caspase-3, also called apopain, CPP32, SREBP cleavage activity 1, yama, and SCA-1 (Human precursor: Swiss-Prot P42574) is a serine protease involved in the apoptosis pathway. Formed from an inactive proenzyme by proteolytic processing to produce 2 subunits that dimerize to form the active enzyme, caspase-3 cleaves and activates caspases 6, 7 and 9. It is the predominant caspase involved in the cleavage of amyloid-beta 4A precursor protein, which is associated with neuronal death in Alzheimer's disease. Fas activates caspase-3 not only by inducing the cleavage of the caspase zymogen to its active subunits, but also by stimulating the denitrosylation of its active-site thiol.

Soluble Elastin Fragments

Elastin is one of the major structural matrix proteins of the arterial wall. Mature elastin is composed of soluble elastin subunits, which are intermolecularly cross-linked into a fibrous network (desmosine and isodesmosine formation) and thus construct a highly polymerized insoluble protein. The main pathological feature of the aortic media in AAD is a higher grade of elastin degradation. Once an initial tear is formed, the dissection tends to expand to the degraded elastin layers, along with an inflammatory infiltrate, a major source of proteolytic enzymes such as elastases and metalloproteinases, which thus dramatically promote the fragmentation process ofthe elastin network in the media. As a result, soluble elastin fragents (SELAF) are released.

Soluble ESAM

Endothelial cell-selective adhesion molecule (Human precursor: Swiss-Prot Q96AP7) is a type I membrane protein localized to tight junctional complexes. Soluble forms may be generated through proteolysis or alternative splicing events, resulting in a non-membrane-bound form containing all or a portion of the extracellular domain of ESAM.

H-FABP

Heart-type fatty acid binding protein is a cytosolic 15 kDa lipid-binding protein involved in lipid metabolism. Heart-type FABP antigen is found not only in heart tissue, but also in kidney, skeletal muscle, aorta, adrenals, placenta, and brain (Veerkamp, J. H. and Maatman, R. G., Prog. Lipid Res. 34:17-52, 1995; Yoshimoto, K. et al., Heart Vessels 10:304-309, 1995). Furthermore, heart-type FABP mRNA can be found in testes, ovary, lung, mammary gland, and stomach (Veerkamp, J. H. and Maatman, R. G., Prog. Lipid Res. 34:17-52, 1995). The normal plasma concentration of FABP is <6 ng/ml (400 pM). The plasma H-FABP concentration is elevated in patients with AMI and unstable angina (Ishii, J. et al., Clin. Chem. 43:1372-1378, 1997; Tsuji, R. et al., Int. J Cardiol. 41:209-217, 1993). Furthermore, H-FABP may be useful in estimating infarct size in patients with AMI (Glatz, J. F. et al., Br. Heart J. 71:135-140, 1994). Myocardial tissue as a source of H-FABP can be confirmed by determining the ratio of myoglobin/FABP (grams/grams). A ratio of approximately 5 indicates that FABP is of myocardial origin, while a higher ratio indicates skeletal muscle sources (Van Nieuwenhoven, F. A. et al., Circulation 92:2848-2854, 1995). Because of the presence of H-FABP in skeletal muscle, kidney and brain, elevations in the plasma H-FABP concentration may be associated with skeletal muscle injury, renal disease, or stroke. H-FABP is released into the bloodstream following cardiac tissue necrosis. The plasma H-FABP concentration can be significantly elevated 1-2 hours after the onset of chest pain, earlier than CK-MB and myoglobin (Tsuji, R. et al., Int. J. Cardiol. 41:209-217, 1993; Van Nieuwenhoven, F. A. et al., Circulation 92:2848-2854, 1995; Tanaka, T. et al., Clin. Biochem. 24:195-201, 1991). Additionally, H-FABP is rapidly cleared from the bloodstream, and plasma concentrations return to baseline after 24 hours after AMI onset (Glatz, J. F. et al., Br. Heart J. 71:135-140, 1994; Tanaka, T. et al., Clin. Biochem. 24:195-201, 1991).

TIMP-1

Tissue inhibitor of metalloproteinases-1 (Human precursor: Swiss-Prot P01033) complexes with, and irreversibly inactivates, metalloproteinases. Known to act on MMP-1, MMP-2, MMP-3, MMP-7, MMP-8, MMP-9, MMP-10, MMP-11, MMP-12, MMP-13 and MMP-16.

Identification of Marker Panels

In accordance with the present invention, there are provided methods and systems for the identification of one or more markers for differential diagnosis and/or risk stratification of a subject. Suitable methods for identifying markers useful for the diagnosis of disease states are described in detail in U.S. Provisional Patent Application No. 60/436,392 filed Dec. 24, 2002, PCT WO2004/058055 published Jul. 15, 2004, U.S. patent application Ser. No. 10/331,127 filed Dec. 27, 2002, and PCT WO2004/059293 published Jul. 15, 2004, each of which is hereby incorporated by reference in its entirety, including all tables, figures, and claims.

One skilled in the art will also recognize that univariate analysis of markers can be performed and the data from the univariate analyses of multiple markers can be combined to form panels of markers to differentiate different disease conditions. Such methods include multiple linear regression, determining interaction terms, stepwise regression, neural net methods, etc.

In developing a panel of markers useful in differential diagnosis, data for a number of potential markers may be obtained from a group of subjects by testing for the presence or level of certain markers. The group of subjects is divided into two sets. The first set includes subjects who have been confirmed as having a disease or, more generally, being in a first condition state. For example, this first set of patients may be those diagnosed with aortic aneurysm or aortic dissection. The confirmation of this condition state may be made through a more rigorous and/or expensive testing to confirm the condition state. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or patients suffering from another unrelated disease for example.

The data obtained from subjects in these sets includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial active region may be initially selected for each marker. The location of the active region may initially be selected at any point, but the selection may affect the optimization process described below. In this regard, selection near a suspected optimal location may facilitate faster convergence of the optimizer. In a preferred method, the active region is initially centered about the center of the overlap region of the two sets of patients. In one embodiment, the active region may simply be a cutoff point. In other embodiments, the active region may have a length of greater than zero. In this regard, the active region may be defined by a center value and a magnitude of length. In practice, the initial selection of the limits of the active region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the active region and another value above the active region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the active region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject (j) is expressed as:
Rⱼ=ΣwᵢIᵢⱼ,
where i is the marker index, j is the subject index, wᵢ is the weighting coefficient for marker i, I is the indicator value to which the marker level for marker i is mapped for subject j, and Σ is the summation over all candidate markers i. The value “R” may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the active region eliminates this concern.

The panel response may also be a general function of several parameters including the marker levels and other factors including, for example, race and gender of the patient, various clinical characteristics, clinical “scores” such as a pulmonary hypertension “Daniel” score, an NIH stroke score, a Sepsis Score of Elebute and Stoner, a Duke Criteria for Infective Endocarditis, a Mannheim Peritonitis Index, an “Apache” score, a Glasgo aneurysm score, etc. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the active region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable that may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and active regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

To allow a determination of test accuracy, a “gold standard” test criterion may be selected which allows selection of subjects into two or more groups for comparison by the foregoing methods. Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures:
at least 75% sensitivity, combined with at least 75% specificity; ROC curve area of at least 0.7, more preferably at least 0.8, even more preferably at least 0.9, and most preferably at least 0.95; and/or
a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

The following table provides a list of preferred markers that may be used individually, or in preferred embodiments combined int multiple marker panels, for performing the methods of the present invention. As discussed herein, the present invention contemplates the inclusion of markers related to each of these markers (as that term is defined herein), as well as various splice variants, polymorphisms, post-translational modifications, etc., known to those of skill in the art, and in particular immunologically detectable fragments of each marker or its biosynthetic parent.

| Marker | Classification |
|---|---|
| Myoglobin | Tissue injury |
| E-selectin | Tissue injury |
| VEGF | Tissue injury |
| EG-VEGF | Tissue injury |
| Cardiac Troponin I (free and/or complexed) | Myocardial injury |
| Cardiac Troponin T (free and/or complexed) | Myocardial injury |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| proANP | Blood pressure regulation |
| NT-proANP | Blood pressure regulation |
| ANP | Blood pressure regulation |
| CNP | Blood pressure regulation |
| Kininogen | Blood pressure regulation |
| CGRP II | Blood pressure regulation |
| urotensin II | Blood pressure regulation |
| proBNP | Blood pressure regulation |
| NT-proBNP | Blood pressure regulation |
| BNP | Blood pressure regulation |
| BNP₇₉₋₁₀₈ | Blood pressure regulation |
| BNP₃₋₁₀₈ | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 | Blood pressure regulation |
| angiotensin 2 | Blood pressure regulation |
| angiotensin 3 | Blood pressure regulation |
| Bradykinin | Blood pressure regulation |
| Tachykinin-3 | Blood pressure regulation |
| calcitonin | Blood pressure regulation |
| Endothelin-2 | Blood pressure regulation |
| Endothelin-3 | Blood pressure regulation |
| Renin | Blood pressure regulation |
| Urodilatin | Blood pressure regulation |
| Urocortin I | Blood pressure regulation |
| Urocortin II | Blood pressure regulation |
| Urocortin III | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Urokinase plasminogen activator surface | Coagulation and hemostasis |
| receptor (uPAR), soluble form | |
| Protein C | Coagulation and hemostasis |
| Soluble Endothelial Protein C Receptor | Coagulation and hemostasis |
| (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| CSRP2 | Vascular tissue |
| Elastin (and/or soluble fragments thereof) | Vascular tissue |
| Endothelial cell-selective adhesion molecule | Vascular tissue |
| (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| smooth muscle myosin heavy chain | Vascular tissue |
| transgelin | Vascular tissue |
| Carboxyterminal propeptide of type I | Collagen synthesis |
| procollagen (PICP) | |
| Collagen carboxyterminal telopeptide (ICTP) | Collagen degradation |
| APRIL (TNF ligand superfamily member 13) | Inflammatory |
| Complement C3a | Inflammatory |
| CCL-5 (RANTES) | Inflammatory |
| CCL-8 (MCP-2) | Inflammatory |
| CCL-19 (macrophage inflammatory | Inflammatory |
| protein-3β) | |
| CCL-20 (MIP-3α) | Inflammatory |
| CCL-23 (MIP-3) | Inflammatory |
| CXCL-5 | Inflammatory |
| CXCL-9 | Inflammatory |
| CXCL-10 | Inflammatory |
| CXCL-13 (small inducible cytokine B13) | Inflammatory |
| CXCL-16 (small inducible cytokine B16) | Inflammatory |
| Glutathione S Transferase | Inflammatory |
| HIF 1 ALPHA | Inflammatory |
| Caspase-1 | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Intracellular adhesion molecule | Inflammatory |
| Lipocalin-2 | Inflammatory |
| Lactate dehydrogenase | Inflammatory |
| MCP-1 | Inflammatory |
| MDA-LDL | Inflammatory |
| MMP-1 | Inflammatory |
| MMP-2 | Inflammatory |
| MMP-3 | Inflammatory |
| MMP-7 | Inflammatory |
| MMP-9 | Inflammatory |
| MMP-10 | Inflammatory |
| TIMP-1 | Inflammatory |
| TIMP-2 | Inflammatory |
| TIMP-3 | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| Transforming growth factor beta | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Pentaxin-related protein PTX3 | Inflammatory |
| Prostaglandin D-synthase | Inflammatory |
| Prostaglandin E2 | Inflammatory |
| RANK ligand | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| Neutrophil elastase | Pulmonary injury |
| KL-6 | Pulmonary injury |
| LAMP 3 | Pulmonary injury |
| LAMP3 | Pulmonary injury |
| Pulmonary surfactant protein A | Pulmonary injury |
| (lung surfactant protein A) | |
| Pulmonary surfactant protein B | Pulmonary injury |
| (lung surfactant protein B) | |
| Pulmonary surfactant protein C | Pulmonary injury |
| (lung surfactant protein C) | |
| Pulmonary surfactant protein D | Pulmonary injury |
| (lung surfactant protein D) | |
| phospholipase D | Pulmonary injury |
| PLA2G5 | Pulmonary injury |
| SFTPC | Pulmonary injury |
| HTI56 | Pulmonary injury |
| HTII280 | Pulmonary injury |
| MAPK10 | Neural tissue injury |
| KCNK4 | Neural tissue injury |
| KCNK9 | Neural tissue injury |
| KCNQ5 | Neural tissue injury |
| 14-3-3 | Neural tissue injury |
| 4.1B | Neural tissue injury |
| APO E4-1 | Neural tissue injury |
| myelin basic protein | Neural tissue injury |
| Atrophin 1 | Neural tissue injury |
| brain Derived neurotrophic factor | Neural tissue injury |
| Brain Fatty acid binding protein | Neural tissue injury |
| brain tubulin | Neural tissue injury |
| CACNA1A | Neural tissue injury |
| Calbindin D | Neural tissue injury |
| Calbrain | Neural tissue injury |
| Carbonic anhydrase XI | Neural tissue injury |
| CBLN1 | Neural tissue injury |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CK-BB | Neural tissue injury |
| CRHR1 | Neural tissue injury |
| C-tau | Neural tissue injury |
| DRPLA | Neural tissue injury |
| GFAP | Neural tissue injury |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIP2 | Neural tissue injury |
| LDH | Neural tissue injury |
| Myelin basic protein | Neural tissue injury |
| NCAM | Neural tissue injury |
| NT-3 | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1, 2, 3, 4 | Neural tissue injury |
| NRG2 | Neural tissue injury |
| PACE4 | Neural tissue injury |
| phosphoglycerate mutase | Neural tissue injury |
| PKC gamma | Neural tissue injury |
| proteolipid protein | Neural tissue injury |
| PTEN | Neural tissue injury |
| PTPRZ1 | Neural tissue injury |
| RGS9 | Neural tissue injury |
| RNA Binding protein Regulatory Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| SREB3 | Neural tissue injury |
| STAC | Neural tissue injury |
| STX1A | Neural tissue injury |
| STXBP1 | Neural tissue injury |
| Syntaxin | Neural tissue injury |
| thrombomodulin | Neural tissue injury |
| transthyretin | Neural tissue injury |
| adenylate kinase-1 | Neural tissue injury |
| BDNF | Neural tissue injury |
| neurokinin A | Neural tissue injury |
| neurokinin B | Neural tissue injury |
| s-acetyl Glutathione | apoptosis |
| cytochrome C | apoptosis |
| Caspase 3 | apoptosis |
| Cathepsin D | apoptosis |
| α-spectrin | apoptosis |
| superoxide dismutase | reactive oxygen species |
| glutathione | reactive oxygen species |
| α-tocopherol, ascorbate | reactive oxygen species |
| inducible nitric oxide synthase | reactive oxygen species |
| lipid peroxidation products | reactive oxygen species |
| nitric oxide | reactive oxygen species |
| myeloperoxidase | reactive oxygen species |
| breath hydrocarbons | reactive oxygen species |

The skilled artisan will recognize that an assay for ubiquitin may be designed that recognizes ubiquitin itself, ubiquitin-protein conjugates, or both ubiquitin and ubiquitin-protein conjugates. For example, antibodies used in a sandwich immunoassay may be selected so that both the solid phase antibody and the labeled antibody recognize a portion of ubiquitin that is available for binding in both unconjugated ubiquitin and ubiquitin conjugates. Alternatively, an assay specific for ubiquitin conjugates of the muscle protein troponin could use one antibody (on a solid phase or label) that recognizes ubiquitin, and a second antibody (the other of the solid phase or label) that recognizes troponin.

The present invention contemplates measuring ubiquitin conjugates of any marker described herein. Preferred ubiquitin-muscle protein conjugates for detection as markers include, but are not limited to, troponin 1-ubiquitin, troponin T-ubiquitin, troponin C-ubiquitin, binary and ternary troponin complex-ubiquitin, actin-ubiquitin, myosin-ubiquitin, tropomyosin-ubiquitin, and a-actinin-ubiquitin.

Assay Measurement Strategies

Numerous methods and devices are well known to the skilled artisan for the detection and analysis of the markers of the instant invention. With regard to polypeptides or proteins such as CK-BB and acidic calponin in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwitch, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of markers using a single test device. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody conjugated to a solid phase and a second antibody conjugated to a signal development element. Such assay devices are configured to perform a sandwich immunoassay for one or more analytes. These assay devices will preferably further comprise a sample application zone, and a flow path from the sample application zone to a second device region comprising the first antibody conjugated to a solid phase.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001, which is hereby incorporated by reference in its entirety. Other measurement strategies applicable to the methods described herein include chromatography (e.g., HPLC), mass spectrometry, receptor-based assays, and combinations of the foregoing.

A panel consisting of the markers referenced above may be constructed to provide relevant information related to differential diagnosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Preferred reagents include antibodies that detect one or more marker of interest (e.g., CK-BB and/or calponin) immobilized on a solid phase, antibodies that detect one or more marker of interest (e.g., CK-BB and/or calponin) conjugated to a detectable label, or most preferably both solid phase and detectably labeled antibodies. Optionally the kits may contain one or more means for using information obtained from immunoassays performed for a marker panel to rule in or out certain diagnoses. Such means may include computer-readable media that provide instructions for converting one or more assay signals into a diagnosis or prognosis, human-readable material such as labels or package inserts containing instructions that an operator may use to manually convert one or more assay signals into a diagnosis or prognosis, etc.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm, Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

### EXAMPLES

The following examples serve to illustrate the present invention. These examples are in no way intended to limit the scope of the invention.

### Example 1

### Blood Sampling

Blood specimens were collected by trained personnel using EDTA as the anticoagulant and centrifuged for greater than or equal to 10 minutes. The plasma component was transferred into a sterile cryovial and frozen at −20° C. or colder. Clinical histories were available for each of the patients to aid in the statistical analysis of the assay data.

### Example 3

### Purified Proteins

Human acidic calponin (amino acids 1-329 of Swiss prot Q15417) was expressed in bacteria. Human CK-BB (Cat # C1124) was purchased from Scripps Laboratories, San Diego, Calif.

### Example 3

### Antibody Development

Antibodies for use in the following biochemical analyses were obtained using phage display techniques. Antibody phage were prepared as generally described in WO 03/068956 and U.S. Pat. No. 6,057,098, the contents of which are incorporated by reference herein in their entirety, including all tables, figures, and claims, using the proteins from Example 2 as immunogens and screening reagents.

### Example 4

### Biochemical Analyses

Markers were measured using standard immunoassay techniques configured to detect a particular marker of interest. These techniques involved the use of antibodies to specifically bind the protein targets. A monoclonal antibody directed against a selected marker was biotinylated using N-hydroxysuccinimide biotin (NHS-biotin) at a ratio of about 5 NHS-biotin moieties per antibody. The antibody-biotin conjugate was then added to wells of a standard avidin 384 well microtiter plate, and antibody conjugate not bound to the plate was removed. This forms the “anti-marker” in the microtiter plate. Another monoclonal antibody directed against the same marker was conjugated to alkaline phosphatase using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Immunoassays were performed on a TECAN Genesis RSP 200/8 Workstation. Biotinylated antibodies were pipetted into microtiter plate wells previously coated with avidin and incubated for 60 min. The solution containing unbound antibody was removed, and the wells washed with a wash buffer, consisting of 20 mM borate (pH 7.42) containing 150 mM NaCl, 0.1% sodium azide, and 0.02% Tween-20. The plasma samples (10 μL) were pipeted into the microtiter plate wells, and incubated for 60 min. The sample was then removed and the wells washed with a wash buffer. The antibody-alkaline phosphatase conjugate was then added to the wells and incubated for an additional 60 min, after which time, the antibody conjugate was removed and the wells washed with a wash buffer. A substrate, (AttoPhos®, Promega, Madison, Wis. or ELISA Aplification System, Invitrogen Corporation, Carlsbad, Calif.) was added to the wells, and the rate of formation of the fluorescent product was related to the concentration of the marker in the patient samples.

### Example 5

### Subject Populations

Subjects are identified below as falling into one of three groups: “AD-” (aortic dissection negative subjects presenting with symptoms consistent with aortic dissection), “AD+” (aortic dissection positive subjects presenting with symptoms consistent with aortic dissection), and “Normal” (normal healthy control subjects not having apparent symptoms of aortic dissection). Samples from AD− and AD+ subjects were obtained as part of a multi-site study, in which subjects presenting for medical evaluation of symptoms were enrolled based upon a suspected aortic dissection etiology for those symptoms. Final diagnosis was made using standard medical techniques for evaluation of aortic dissection. Acidic calponin and CK-BB were measured on samples taken at the time of presentation. Samples from normal subjects were obtained from a commercial source.

There are two classifications systems for aortic dissection: the Stanford classification and the DeBakey classification. The Stanford classification divides the dissections into 2 types, type A and type B. Type A involves the ascending aorta and typically requires surgery while type B is less severe and involves the descending aorta.

### Example 6

### CK-BB as a Marker of Aortic Dissection

A preferred marker of aortic dissection could be expected to distinguish AD+ subjects from normal subjects (that is, subjects that are not symptom mimics for AD). To assess the ability of CK-BB to distinguish AD+ subjects from normal subjects at the time of presentation, samples were measured as described in the foregoing examples. Quoted concentrations are in ng/ml. As noted, AD+ subjects were also subdivided into Type A and Type B aortic dissection, according to the Stanford Classification. The following table summarizes the results obtained:

| | Normal | AD+ | AD+ Type A | AD+ Type B |
|---|---|---|---|---|
| N | 277 | 36 | 18 | 16 |
| Concentration, 25th | 0.13 | 0.13 | 0.14 | 0.13 |
| percentile | | | | |
| Concentration, median | 0.13 | 0.50 | 0.60 | 0.43 |
| Concentration, mean | 0.38 | 2.11 | 1.27 | 3.28 |
| Concentration, 75th % tile | 0.17 | 1.09 | 0.98 | 1.37 |

Based on the ROC analysis of this data, CK-BB is able to distinguish normal subjects from AD+ subjects, including both Type A and Type B AD+ subjects, in a statistically significant manner. The following table summarizes the ROC analysis:

| | AD− vs | AD− vs type | AD− vs. type |
|---|---|---|---|
| | AD+ | A AD+ | B AD+ |
| N (normal) | 277 | 277 | 277 |
| N (AD+) | 36 | 18 | 16 |
| ROC area | 0.76 | 0.78 | 0.76 |
| P | <0.001 | <0.001 | <0.001 |

### Example 7

### Acidic Calponin as a Marker of Aortic Dissection

A particularly preferred marker of aortic dissection could be expected to distinguish AD+subjects from mimics. A “mimic” of aortic dissection as that term is used herein refers to subjects presenting to a medical facility for evaluation of symptoms that are consistent with one or more conditions that include aortic dissection; another way of stating this is that aortic dissection is within the differential diagnosis of the symptoms presented. As discussed herein, medical personnel are often asked to identify the cause of symptoms from amongst the conditions within the differential diagnosis for those symptoms. To assess the ability of acidic calponin to distinguish AD+ subjects from AD− mimics at the time of presentation, samples were measured as described in the foregoing examples. Quoted concentrations are in ng/ml. As noted, AD+ subjects were also subdivided into Type A and Type B aortic dissection, according to the Stanford Classification. The following table summarizes the results obtained:

| | AD− | AD+ | AD+ Type A | AD+ Type B |
|---|---|---|---|---|
| N | 51 | 37 | 22 | 13 |
| Concentration, 25th percentile | 1.16 | 2.03 | 2.29 | 1.01 |
| Concentration, median | 1.92 | 2.95 | 3.68 | 2.03 |
| Concentration, mean | 3.36 | 6.10 | 8.55 | 2.44 |
| Concentration, 75th % tile | 2.66 | 4.38 | 8.74 | 3.64 |

Based on the ROC analysis of this data, acidic calponin is able to distinguish AD− subjects from AD+ subjects, and particularly Type A AD+ subjects, in a statistically significant manner. The following table summarizes the ROC analysis:

| | | AD− vs type | AD− vs. type |
|---|---|---|---|
| | AD− vs AD+ | A AD+ | B AD+ |
| N (AD−) | 51 | 51 | 51 |
| N (AD+) | 37 | 22 | 13 |
| ROC area | 0.69 | 0.78 | 0.53 |
| P | 0.003 | <0.001 | >0.05 |

### Example 8

### Additional Aortic Dissection Markers

In addition to CK-BB and acidic calponin, assays were configured to detect the following markers in order to assess the ability of such assays to distinguish aortic dissection. The Swiss-Prot entry number for the human precursor and measured units for each marker are in parentheses: basic calponin (P51911, ng/mL), caldesmon (Q05682, pg/mL), caspase-3 (P42574, ng/mL), soluble elastin fragments (“sELAF”) (P15502, ng/mL), soluble endothelial cell-selective adhesion molecule extracellular domain (“sESAM”) (Q96AP7, ng/mL), heart-type fatty acid binding protein (“hFABP”) (P05413, ng/mL), and TIMP-1 (P01033, μg/mL). The following table summarizes the results obtained:

| | | | AD+ | AD+ |
|---|---|---|---|---|
| | Normals | AD+ | Type A | Type B |
| Basic Calponin | | | | |
| N | 282 | 79 | 47 | 29 |
| Concentration, mean | 44.1 | 242.7 | 286.7 | 186.5 |
| Concentration, median | 31.7 | 144.3 | 145.9 | 144.3 |
| Concentration, 95th % tile | 124.4 | 1090 | 1208 | 559.9 |
| Caldesmon | | | | |
| N | 176 | 79 | 47 | 29 |
| Concentration, mean | 64.6 | 542.2 | 615.0 | 470.9 |
| Concentration, median | 24.2 | 390.0 | 558.2 | 240.2 |
| Concentration, 95th % tile | 261.3 | 1610 | 1594 | 1634 |
| Caspase-3 | | | | |
| N | 23 | 45 | 26 | 17 |
| Concentration, mean | 2.68 | 9.66 | 10.8 | 8.52 |
| Concentration, median | 0.98 | 5.26 | 6.04 | 3.50 |
| Concentration, 95th % tile | 5.47 | 40.0 | 40.0 | 39.1 |
| sELAF | | | | |
| N | 282 | 79 | 47 | 29 |
| Concentration, mean | 11.8 | 25.1 | 27.7 | 22.6 |
| Concentration, median | 9.94 | 20.2 | 23.1 | 15.2 |
| Concentration, 95th % tile | 24.1 | 65.9 | 77.2 | 65.5 |
| sESAM | | | | |
| N | 176 | 79 | 47 | 29 |
| Concentration, mean | 43.3 | 74.1 | 72.5 | 75.4 |
| Concentration, median | 43.9 | 69.0 | 69.8 | 66.3 |
| Concentration, 95th % tile | 55.5 | 139.4 | 114.8 | 139.4 |
| hFABP | | | | |
| N | 282 | 32 | 17 | 13 |
| Concentration, mean | 4.73 | 45.9 | 76.2 | 11.9 |
| Concentration, median | 4.28 | 8.98 | 12.3 | 5.44 |
| Concentration, 95th % tile | 8.50 | 318.6 | 448.6 | 82.0 |
| TIMP-1 | | | | |
| N | 282 | 32 | 17 | 13 |
| Concentration, mean | 0.40 | 0.36 | 0.40 | 0.31 |
| Concentration, median | 0.24 | 0.34 | 0.36 | 0.32 |
| Concentration, 95th % tile | 0.33 | 0.52 | 0.98 | 0.39 |

| ROC areas | | Type A AD+ vs | Type B AD+ vs |
|---|---|---|---|
| (p value): | AD+ vs normal | normal | normal |
| Basic Calponin | 0.84 (<0.001) | 0.85 (<0.001) | 0.84 (<0.001) |
| Caldesmon | 0.91 (<0.001) | 0.93 (<0.001) | 0.87 (<0.001) |
| Caspase-3 | 0.82 (<0.001) | 0.83 (<0.001) | 0.80 (<0.001) |
| sELAF | 0.76 (<0.001) | 0.81 (<0.001) | 0.71 (<0.001) |
| sESAM | 0.89 (<0.001) | 0.88 (<0.001) | 0.87 (<0.001) |
| hFABP | 0.76 (<0.001) | 0.86 (<0.001) | 0.61 (0.20) |
| TIMP-1 | 0.84 (<0.001) | 0.87 (<0.001) | 0.81 (<0.001) |

### Example 9

### Further Marker Studies

Assays configured to detect the following markers were used in order to assess the ability of such assays to distinguish aortic dissection. The following table summarizes the results obtained:

| | | | | |
|---|---|---|---|---|
| MARKER | N | Average | Median | StdDev |

Normals

| | | | | |
|---|---|---|---|---|
| BNP pg/ml | 204 | 8.36 | 5.00 | 8.00 |
| CKMB ng/ml | 204 | 1.67 | 1.00 | 3.48 |
| Myoglobin ng/ml | 204 | 62.33 | 49.81 | 53.18 |
| cTnI ng/ml | 204 | 0.05 | 0.05 | 0.01 |
| Acidic Calponin | 218 | 1.19 | 1.01 | 0.92 |
| ng/ml | | | | |
| Basic Calponin ng/ml | 282 | 44.09 | 31.74 | 41.81 |
| Caldesmon pg/ml | 184 | 74.14 | 25.52 | 141.43 |
| Caspase-3 ng/ml | 23 | 2.68 | 0.98 | 6.77 |
| CK-BB ng/ml | 282 | 0.20 | 0.13 | 0.24 |
| D-Dimer ng/ml | 204 | 165.46 | 88.56 | 370.52 |
| sELAF ng/ml | 282 | 11.78 | 9.94 | 9.20 |
| sESAM ng/ml | 176 | 43.25 | 43.88 | 11.06 |
| sJAM2 ng/ml | 218 | 218.49 | 56.84 | 555.54 |
| sJAM3 ng/ml | 265 | 3.14 | 2.25 | 8.50 |
| Neutral Calponin | 230 | 12.61 | 5.00 | 64.72 |
| ng/ml | | | | |
| TIMP-1 ug/ml | 282 | 0.33 | 0.24 | 0.68 |
| hFABP ng/ml | 282 | 4.73 | 4.28 | 2.53 |

AD+ (Types A & B)

| | | | | |
|---|---|---|---|---|
| BNP pg/ml | 24 | 327.54 | 88.73 | 694.04 |
| CKMB ng/ml | 24 | 3.91 | 1.23 | 7.00 |
| Myoglobin ng/ml | 24 | 186.17 | 102.79 | 167.58 |
| cTnI ng/ml | 24 | 1.34 | 0.05 | 6.11 |
| Acidic Calponin | 47 | 3.64 | 2.39 | 5.37 |
| ng/ml | | | | |
| Basic Calponin ng/ml | 47 | 202.32 | 130.13 | 268.69 |
| Caldesmon pg/ml | 47 | 613.23 | 402.03 | 601.23 |
| Caspase-3 ng/ml | 24 | 11.40 | 5.76 | 12.96 |
| CK-BB ng/ml | 47 | 1.37 | 0.45 | 3.44 |
| D-Dimer ng/ml | 24 | 3714.70 | 4431.21 | 1484.87 |
| sELAF ng/ml | 47 | 24.44 | 20.04 | 18.53 |
| sESAM ng/ml | 47 | 77.59 | 69.84 | 40.36 |
| sJAM2 ng/ml | 47 | 97.68 | 24.48 | 154.63 |
| sJAM3 ng/ml | 23 | 5.22 | 3.93 | 6.62 |
| Neutral Calponin | 47 | 14.93 | 5.43 | 22.65 |
| ng/ml | | | | |
| TIMP-1 ug/ml | 47 | 0.43 | 0.40 | 0.24 |
| hFABP ng/ml | 19 | 34.60 | 7.62 | 57.73 |

Type A Aortic Dissection

| | | | | |
|---|---|---|---|---|
| BNP pg/ml | 14 | 426.28 | 58.81 | 896.44 |
| CKMB ng/ml | 14 | 5.58 | 2.24 | 8.85 |
| Myoglobin ng/ml | 14 | 218.13 | 107.32 | 193.41 |
| cTnI ng/ml | 14 | 2.25 | 0.05 | 7.99 |
| Acidic Calponin | 31 | 4.48 | 2.64 | 6.44 |
| ng/ml | | | | |
| Basic Calponin ng/ml | 31 | 224.40 | 106.36 | 315.17 |
| Caldesmon pg/ml | 31 | 595.47 | 543.34 | 484.44 |
| Caspase-3 ng/ml | 14 | 10.06 | 5.76 | 11.27 |
| CK-BB ng/ml | 31 | 1.87 | 0.74 | 4.16 |
| D-Dimer ng/ml | 14 | 3599.04 | 4067.80 | 1493.79 |
| sELAF ng/ml | 31 | 26.51 | 22.83 | 20.97 |
| sESAM ng/ml | 31 | 75.74 | 67.91 | 39.72 |
| sJAM2 ng/ml | 31 | 113.68 | 24.48 | 181.55 |
| sJAM3 ng/ml | 13 | 6.39 | 4.10 | 8.64 |
| Neutral Calponin | 31 | 17.55 | 5.00 | 27.11 |
| ng/ml | | | | |
| TIMP-1 ug/ml | 31 | 0.46 | 0.41 | 0.28 |
| hFABP ng/ml | 12 | 50.59 | 9.59 | 68.45 |

Type B Aortic Dissection

| | | | | |
|---|---|---|---|---|
| BNP pg/ml | 10 | 189.30 | 141.83 | 183.90 |
| CKMB ng/ml | 10 | 1.58 | 1.01 | 1.26 |
| Myoglobin ng/ml | 10 | 141.43 | 99.15 | 117.99 |
| cTnI ng/ml | 10 | 0.05 | 0.05 | 0.00 |
| Acidic Calponin | 15 | 1.96 | 2.04 | 1.10 |
| ng/ml | | | | |
| Basic Calponin ng/ml | 15 | 159.24 | 140.30 | 145.54 |
| Caldesmon pg/ml | 15 | 687.55 | 240.15 | 809.59 |
| Caspase-3 ng/ml | 10 | 13.26 | 6.15 | 15.48 |
| CK-BB ng/ml | 15 | 0.42 | 0.30 | 0.45 |
| D-Dimer ng/ml | 10 | 3876.63 | 4842.55 | 1536.73 |
| sELAF ng/ml | 15 | 20.42 | 16.85 | 12.60 |
| sESAM ng/ml | 15 | 80.89 | 72.80 | 44.14 |
| sJAM2 ng/ml | 15 | 69.78 | 31.45 | 78.10 |
| sJAM3 ng/ml | 9 | 3.37 | 3.73 | 1.54 |
| Neutral Calponin | 15 | 10.16 | 5.95 | 7.97 |
| ng/ml | | | | |
| TIMP-1 ug/ml | 15 | 0.36 | 0.37 | 0.13 |
| hFABP ng/ml | 7 | 7.18 | 5.44 | 4.45 |

Based on the ROC analysis of this data, each of the markers tested is able to distinguish AD− subjects from AD+ subjects, and particularly Type A AD+ subjects, in a statistically significant manner. The following table summarizes the ROC analysis:

| MARKER | #Diseased | #NonDiseased | ROC AUC | p-value |
|---|---|---|---|---|
| | Diseased = AD+, Type A or B (0-24 h) | | | |
| | Non-Diseased = Normal | | | |
| BNP | 24 | 204 | 0.91 | <0.01 |
| CKMB | 24 | 204 | 0.66 | 0.01 |
| Myo | 24 | 204 | 0.88 | <0.01 |
| TnI | 24 | 204 | 0.57 | 0.23 |
| Acidic Calponin | 47 | 218 | 0.83 | <0.01 |
| Basic Calponin | 47 | 282 | 0.85 | <0.01 |
| Caldesmon | 47 | 184 | 0.88 | <0.01 |
| Caspase-3 | 24 | 23 | 0.88 | <0.01 |
| CK-BB | 47 | 282 | 0.79 | <0.01 |
| D-Dimer | 24 | 204 | 1.00 | <0.01 |
| sELAF | 47 | 282 | 0.77 | <0.01 |
| sESAM | 47 | 176 | 0.90 | <0.01 |
| sJAM2 | 47 | 218 | 0.37 | 0.01 |
| sJAM3 | 23 | 265 | 0.85 | <0.01 |
| Neutral Calponin | 47 | 230 | 0.65 | <0.01 |
| TIMP-1 | 47 | 282 | 0.87 | <0.01 |
| hFABP | 19 | 282 | 0.80 | <0.01 |
| | Diseased = Type A AD+ (0-24 h) | | | |
| | Non-Diseased = Normal | | | |
| BNP | 14 | 204 | 0.86 | <0.01 |
| CKMB | 14 | 204 | 0.73 | <0.01 |
| Myo | 14 | 204 | 0.85 | <0.01 |
| TnI | 14 | 204 | 0.64 | 0.09 |
| Acidic Calponin | 31 | 218 | 0.86 | <0.01 |
| Basic Calponin | 31 | 282 | 0.85 | <0.01 |
| Caldesmon | 31 | 184 | 0.91 | <0.01 |
| Caspase-3 | 14 | 23 | 0.88 | <0.01 |
| CK-BB | 31 | 282 | 0.82 | <0.01 |
| D-Dimer | 14 | 204 | 1.00 | <0.01 |
| sELAF | 31 | 282 | 0.77 | <0.01 |
| sESAM | 31 | 176 | 0.90 | <0.01 |
| sJAM2 | 31 | 218 | 0.38 | 0.03 |
| sJAM3 | 13 | 265 | 0.89 | <0.01 |
| Neutral Calponin | 31 | 230 | 0.63 | 0.02 |
| TIMP-1 | 31 | 282 | 0.89 | <0.01 |
| hFABP | 12 | 282 | 0.88 | <0.01 |
| | Diseased = Type B (0-24 h) Non-Diseased = Normal | | | |
| BNP | 10 | 204 | 0.97 | <0.01 |
| CKMB | 10 | 204 | 0.56 | 0.50 |
| Myo | 10 | 204 | 0.91 | <0.01 |
| TnI | 10 | 204 | 0.49 | 0.92 |
| Acidic Calponin | 15 | 218 | 0.77 | <0.01 |
| Basic Calponin | 15 | 282 | 0.84 | <0.01 |
| Caldesmon | 15 | 184 | 0.84 | <0.01 |
| Caspase-3 | 10 | 23 | 0.88 | <0.01 |
| CK-BB | 15 | 282 | 0.73 | <0.01 |
| D-Dimer | 10 | 204 | 1.00 | <0.01 |
| sELAF | 15 | 282 | 0.74 | <0.01 |
| sESAM | 15 | 176 | 0.88 | <0.01 |
| sJAM2 | 15 | 218 | 0.38 | 0.11 |
| sJAM3 | 9 | 265 | 0.77 | 0.01 |
| Neutral Calponin | 15 | 230 | 0.70 | 0.01 |
| TIMP-1 | 15 | 282 | 0.82 | <0.01 |
| hFABP | 7 | 282 | 0.67 | 0.11 |

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for assigning a diagnosis to a subject suspected of a disease of the aorta or its branches, or a prognosis to a subject diagnosed with a disease of the aorta or its branches, comprising:
performing one or more assays that detect the presence or amount of one or more markers independently selected from the group consisting of markers related to blood pressure regulation, markers related to inflammation, markers related to vascular tissue, markers related to apoptosis, markers related to reactive oxygen species, markers related to myocardial injury, and markers related to coagulation and hemostasis; and
determining the presence or absence of a disease of the aorta or its branches in said subject, or to a likelihood of an outcome in said subject, from the results obtained from said assay(s).

2. A method according to claim 1, wherein said performing step comprises performing one or more assays that detect the presence or amount of one or more markers selected from the group consisting of creatine kinase-BB (CK-BB), creatine kinase-MB (CK-MB), acidic calponin, basic calponin, neutral calponin, soluble junction adhesion molecule-I (sJAM1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

3. A method according to claim 2, wherein the determining step comprises:
calculating a concentration of one or more markers selected from the group consisting of creatine kinase-BB (CK-BB), creatine kinase-MB (CK-MB), acidic calponin, basic calponin, neutral calponin, soluble junction adhesion molecule-1 (sJAM 1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T based on detectable signal(s) from the assay(s) performed; and
comparing each calculated concentration to a corresponding marker threshold level that is indicative of the presence or absence of an aortic aneurysm or that is indicative of the presence or absence of an aortic dissection, or
calculating a single value that is a function of each calculated concentration and comparing that single value to a threshold level that is indicative of the presence or absence of an aortic aneurysm or that is indicative of the presence or absence of an aortic dissection.

4. A method according to claim 2, wherein the method further comprises performing one or more additional assays that detect one or more additional markers on a sample obtained from said subject, and wherein the presence or absence of a disease of the aorta or its branches in said subject, or to a likelihood of an outcome in said subject, is determined from the results obtained from said assay(s) and from said additional assay(s).

5. A method according to claim 4, wherein the additional assays detect at least one marker related to coagulation and hemostasis.

6. A method according to claim 4, wherein the additional assays detect at least one marker related to blood pressure regulation.

7. A method according to claim 4, wherein the additional assays detect at least one marker related to inflammation.

8. A method according to claim 4, wherein the additional assays detect at least one marker related to vascular tissue.

9. A method according to claim 4, wherein the additional assays detect at least one marker related to apoptosis.

10. A method according to claim 4, wherein the additional assays detect at least one marker related to reactive oxygen species.

11. A method according to claim 4, wherein the additional assays detect at least one marker related to myocardial injury.

12. A method according to claim 1, wherein the sample is from a human.

13. A method according to claim 1, wherein the sample is selected from the group consisting of blood, serum, and plasma.

14. A method according to claim 1, wherein the assay(s) performed is(are) immunoassay(s).

15. A method according to claim 1, wherein said performing step comprises performing an assay that detects the presence or amount of one or more of B-type natriuretic peptide, NT-proBNP, proBNP, BNP₇₉₋₁₀₈, or BNP₃₋₁₀₈.

16. A method according to claim 1, wherein said performing step comprises performing an assay that detects D-dimer.

17. A method according to claim 1, wherein the wherein said performing step comprises performing an assay that detects free cardiac troponin I, complexed cardiac troponin I, free and complexed cardiac troponin I, free cardiac troponin T, complexed cardiac troponin T, free and complexed cardiac troponin T, or total cardiac troponin.

18. A method according to claim 1, wherein said disease of the aorta or its branches is aortic aneurysm.

19. A method according to claim 1, wherein said disease of the aorta or its branches is aortic dissection.

20. A kit for performing a test to diagnose a disease of the aorta or its branches, or to determine a prognosis associated with a disease of the aorta or its branches, comprising:
one or more antibodies, each said antibody binding for detection a marker independently selected from the group consisting of markers related to blood pressure regulation, markers related to inflammation, markers related to vascular tissue, markers related to apoptosis, markers related to reactive oxygen species, markers related to myocardial injury, and markers related to coagulation and hemostasis; and
instructions for diagnosing a disease of the aorta or its branches, or for determining a prognosis associated with a disease of the aorta or its branches based on assay results obtained using said antibod(y)(ies).

21. A kit according to claim 20, wherein said one or more antibodies bind for detection one or more markers selected from the group consisting of creatine kinase-BB (CK-BB), creatine kinase-MB (CK-MB), acidic calponin, basic calponin, neutral calponin, soluble junction adhesion molecule-I (sJAM 1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

22. A kit according to claim 20, wherein said instructions are provided on a computer-readable medium.

23. A kit according to claim 20, wherein said instructions are provided on a label or package insert.

24. A kit according to claim 20, wherein (a), (b) or (c) is provided in an assay device comprising a sample application zone and a flow path from said sample application zone to a second device region comprising said one or more antibodies bound thereto.

25. A kit according to claim 24, wherein flow of sample along the flow path is driven by a passive force selected from the group consisting of capillary force, hydrostatic force, and a combination of such passive forces, by application of an active force selected from the group consisting of mechanical pumping force, electroosmotic pumping force, centrifugal force, increased air pressure, and a combination of one or more such active forces, or by a combination of said active and passive forces.

26. A method according to claim 1, wherein said performing step comprises performing at least two assays.

27. A method according to claim 1, wherein said performing step comprises performing at least three assays.

28. A method according to claim 1, wherein said performing step comprises performing at least four assays.

29. A method according to claim 1, wherein said performing step comprises performing at least five assays.

30. A method according to claim 1, wherein said performing step comprises performing at least six assays.

31. A method according to claim 1, wherein said performing step comprises performing at least two assays that detect the presence or amount of markers selected from the group consisting of creatine kinase-BB (CK-BB), creatine kinase-MB (CK-MB), acidic calponin, basic calponin, neutral calponin, soluble junction adhesion molecule-1 (sJAM1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

32. A method according to claim 1, wherein said performing step comprises performing at least three assays that detect the presence or amount of markers selected from the group consisting of creatine kinase-BB (CK-BB), creatine kinase-MB (CK-MB), acidic calponin, basic calponin, neutral caiponin, soluble junction adhesion molecule-1 (sJAM1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

33. A method according to claim 1, wherein said performing step comprises performing at least four assays that detect the presence or amount of markers selected from the group consisting of creatine kinase-BB (CK-BB), creatine kinase-MB (CK-MB), acidic calponin, basic calponin, neutral calponin, soluble junction adhesion molecule-1 (sJAM1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

34. A method according to claim 1, wherein said performing step comprises performing at least five assays that detect the presence or amount of markers selected from the group consisting of creatine kinase-BB (CK-BB), creatine kinase-MB (CK-MB), acidic calponin, basic calponin, neutral calponin, soluble junction adhesion molecule-1 (sJAM1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin- 1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

35. A method according to claim 1, wherein said performing step comprises performing at least six assays that detect the presence or amount of markers selected from the group consisting of creatine kinase-BB (CK-BB), creatine kinase-MB (CK-MB), acidic calponin, basic calponin, neutral calponin, soluble junction adhesion molecule-1 (sJAM1), soluble junction adhesion molecule-2 (sJAM2), soluble junction adhesion molecule-3 (sJAM3), B-type natriuretic peptide (BNP), NT-proBNP, proBNP, BNP₇₉₋₁₀₈, BNP₃₋₁₀₈, caldesmon, caspase-3, D-dimer, soluble elastin fragments, soluble endothelial cell-selective adhesion molecule (sESAM), fibrillin-1, heart-type fatty acid binding protein, MMP-9, myeloperoxidase, myoglobin, smooth muscle myosin, smooth muscle myosin heavy chain, TIMP-1, free cardiac troponin I, complexed cardiac troponin I, free and complexed cardiac troponin I, free cardiac troponin T, complexed cardiac troponin T, and free and complexed cardiac troponin T.

36. A method according to claim 1, wherein said performing step comprises performing at least one assay that detect the presence or amount of one or more markers selected from the group consisting of BNP, NT-proBNP, proBNP, creatine kinase-BB (CK-BB), creatine kinase-MB (CK-MB), acidic calponin, basic calponin, neutral calponin, soluble junction adhesion molecule-2 (sJAM2), soluble junction adhesion molecule-3 (sJAM3), caldesmon, caspase-3, sESAM, heart-type fatty acid binding protein, and TIMP-1.

37. A method according to claim 1, wherein said performing step comprises performing at least one assay that detect the presence or amount of one or more markers selected from the group consisting of creatine kinase-BB (CK-BB), acidic calponin, basic calponin, neutral calponin, sJAM2, sJAM3, caldesmon, caspase-3, and sESAM.


SEQUENCE LISTING (2 sequences)

SEQ ID NO: 1 - protein, 108 aa, Artificial Sequence
  Description of Artificial Sequence Synthetic protein construct
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Artificial Sequence
  Description of Artificial Sequence Synthetic protein construct
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH